(19) United States
(12) Patent Application Publication
(10) Pub. No.: US 2012/0016366 A1
(43) Pub. Date: Jan. 19, 2012

(54) Proximal Radius Locking Plate

(76) Inventor: W. Andrew Eglseder, Owings Mills, MD (US)

(21) Appl. No.: 13/257,655

(22) PCT Filed: Apr. 5, 2010
(86) PCT No.: PCT/US2010/029894

§ 371 Date: Sep. 20, 2011

Related U.S. Application Data
(60) Provisional application No. 61/167,684, filed on Apr. 8, 2009.

(51) Int. Cl. A61B 17/80

(52) U.S. Cl. 606/70; 606/286

(57) ABSTRACT

The invention provides a proximal radius locking plate having a body comprising an elongate shaft portion and a head portion. The plate may have a gentle S-shape and a proximal twist. The shaft portion is adapted for receiving bone screws to fix the bone plate to a shaft of the radius. The head portion includes a first head section and a second head section. The first and second head sections extend laterally away from a longitudinal axis of the shaft portion in generally opposite directions, generally forming a T-shape. The first and second head sections are configured and arranged to secure a plurality of bone screws divergently implanted in the head of proximal radius. The shaft portion is thinner proximally in order to preserve the biceps tendon. Moreover, the plate of the present invention can be used as a guide in reducing the fracture prior to fixation since the plate has a precontoured shape matching the shape of an unfractured proximal radius.

### TECHNICAL FIELD

The present invention relates generally the treatment of fractures and to devices used for stabilizing bone fractures, and more particularly the invention relates to bone plates and even more particularly the invention relates to a bone plate for fixating fractures of a proximal radius bone.

### BACKGROUND ART

The radius is one of two long bones found in the human forearm. The radius, like other bones, is susceptible to a variety of fractures and other dislocations. For example, fractures of the radius are a common result of forward falls, with the palms facing downward. In such falls, force exerted on the hands and wrist at impact frequently produces displacement of one or more bone fragments created distal to the fracture site. Fractures of the proximal radius typically result from traumatic injuries such as sporting accidents, and can be more frequent with age due to bone loss. Fracture to the metaphyseal portion of such a long bone can be difficult to treat. Improper treatment can result in deformity and long-term discomfort. In particular, fractures of the radial head can lead to joint pain and elbow instability.

Alignment and fixation of a metaphyseal fracture (occurring at the extremity of a shaft of a long bone) are typically performed by one of several methods: casting, external fixation, pinning, and plating. Casting is non-invasive, but may not be able to maintain alignment of the fracture where many bone fragments exist. Therefore, as an alternative, external fixators may be used. External fixators utilize a method known as ligamentotaxis, which provides distraction forces across the joint and permits the fracture to be aligned based upon the tension placed on the surrounding ligaments. However, while external fixators can maintain the position of the wrist bones, it may nevertheless be difficult in certain fractures, such as fractures of the elbow, to first provide the bones in proper alignment. In addition, external fixators are often not suitable for fractures resulting in multiple bone fragments. Pinning with K-wires (Kirschner wires) is an invasive procedure whereby pins are positioned into the various fragments. This is a difficult and time-consuming procedure that provides limited fixation if the bone is comminuted or osteoporotic. Plating utilizes a stabilizing metal plate typically placed against the dorsal side of a bone and screws extending from the plate into holes drilled in the bone fragments to provide stabilized fixation of the fragments. However, many currently available plate systems fail to provide desirable alignment and stabilization.

Fractures of the proximal radius may be treated by exposing the fracture site and reducing the bone fracture, and then placing a plate or other means onto the bone to fixate the fracture for healing in the reduced position. Reducing the fracture includes realigning and positioning the fractured portions of the bone to their original position or similar stable position. Fixating the fracture includes positioning a plate over the fractured portions and securing the plate onto the fractured bone and adjacent nonfractured bone with bone screws.

Where a single fracture occurs, the original position of the fractured portion can usually be easily recognized, and the unfractured portion functions as a guide or reference point for reducing and fixating the fractured portion to the unfractured bone portion. However, this challenge is more acute with multiple part fractures and fracture dislocations since no single portion of the bone remains unfractured. Therefore, no single portion of the fractured bone can act as a stable guide or reference to insure the return of the fractured portions to their proper position, and to remain stable to enable proper reduction and fixation of the multiple fractured portions at the same time.

In some instances, where there are multiple part fractures and a combined fracture and dislocation of the proximal radius (from the joint space), the common techniques of open reduction and internal fixation are particularly inadequate. In these instances, the conventional response is to replace the fractured bone portion with a prosthetic implant, e.g. artificial proximal radius, instead of reducing and fixating the fractured area. Examples of a radial head prosthesis are shown in U.S. Patent Application Publication Nos. 2003/0212457 and 2001/0037154, and in U.S. Pat. Nos. 6,656,225; 6,361,563; 6,270,529; and 6,217,616. This approach has been favored in treating multiple part fractures and fracture dislocations due to the difficulty of managing the reduction with complicated fracture patterns and the difficulty of adapting available plates for fixation in these situations. Nevertheless, because the artificial replacement of a proximal radius requires significantly more surgery and expense, and since it is preferable to preserve the natural bones whenever possible, it would be desirable to treat these more difficult cases with open reduction and internal fixation with a bone plate.

Examples of a bone fixation plates are described in U.S. Pat. Nos. 6,712,820 and 7,294,130, both to Orbay, and U.S. Pat. No. 6,221,073, to Weiss et al., all of which disclose a bone plate apparatus for use with fixation of wrist fractures; that is, each is directed to the distal end of the radius bone, which has a very different structure than the proximal end. The plate is positioned on the dorsal or volar side of the radius and a plurality of bone screws secure the plate along a non-fractured portion of the bone.

Another example of a bone fixation device of the prior art is described in U.S. Pat. No. 6,283,969, to Grusin et al., which discloses a distal radial plate having a relatively wide T-shape. The longitudinal sections of the plate contain a plurality of recessed holes for insertion of a bone screw. The plate is designed to be bent along its longitudinal or transverse segments of the plate.

U.S. Pat. No. 6,096,040, to Esser, discloses a bone plate especially adapted for repairs of the proximal humerus bone. The plate is configured and arranged to match the contour of an unfractured proximal humerus. A plurality of holes and slots is provided to secure multiple fractures of the humeral head.

U.S. Pat. No. 7,354,441, to Frigg, and U.S. Patent Application Publication No. 2007/0233114, by Bouman, describe bone plates having particularly configured holes and slots for attaching and locking bones screws to the plate and to the bone under repair. In some cases, the slot may be configured to engage threads on the head of a bone screw; alternatively, the slot may be configured to cooperate with the head of a different style bone screw.

None of the prior art discloses a bone plate especially configured for the proximal end of the radius and the radial head. It would be desirable to provide a bone fixation device for use with the proximal end of the radius and the radial head. Moreover, such a fixation device should provide desirable alignment and stabilization of the bone structure proximate the articular surface of the proximal radius.

### DISCLOSURE OF INVENTION

Accordingly, it is an object of the present invention to provide a proximal radius locking plate that avoids the disadvantages of the prior art.

It is an object of the present invention to provide a proximal radius locking plate that enables fixation of radial head fractures and fractures of the proximal radius. A related object of the present invention is to provide a proximal radius locking plate that is held in place by locking screws.

It is another object of the present invention to provide a proximal radius locking plate configured and arranged to match the contour of a healthy unfractured proximal radius. A related object of the present invention is to provide a proximal radius locking plate having a gentle S-shape. A further related object of the present invention is to provide a proximal radius locking plate having a proximal twist.

Another object of the present invention is to provide a proximal radius locking plate sized and configured to avoid impingement on the bicep tendon.

These and other objects of the present invention are accomplished by providing a proximal radius locking plate having a body comprising an elongate shaft portion and a head portion. The shaft portion is adapted for receiving bone screws to fix the bone plate to a shaft of the radius. The head portion includes a first head section and a second head section. The first and second head sections extend laterally away from a longitudinal axis of the shaft portion in generally opposite directions, generally forming a T-shape. The first and second head sections are configured and arranged to secure a plurality of bone screws divergently implanted in the head of the proximal radius. The configuration of the head portion permits the bone plate to be used immediately without having to cut portions of the head portion, as is typically the case with conventional cloverleaf and AO-T plates. The shaft portion is thinner proximally in order to preserve the bicep tendon. Moreover, the plate of the present invention can be used as a guide in reducing the fracture prior to fixation since the plate has a precontoured shape matching the shape of an unfractured proximal radius.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 is a posterior plan view of a proximal radius locking plate according to an embodiment of the present invention.

FIG. 1a shows a cross section of the locking plate taken along the line A-A of FIG. 1.

FIG. 2 is anterior plan view of a proximal radius locking plate according to an embodiment of the present invention.

### BEST MODE(S) FOR CARRYING OUT THE INVENTION

The invention provides a bone plate especially adapted for fixing fractures of the proximal radius. The bone plate is pre-shaped to match the contour of the anatomic shape of an unfractured proximal radius in order to assist in reduction of complicated fractures and to avoid bending and cutting of a conventional plate, which can result in a fatigue failure and fracture of the plate.

The plate of the present invention is generally T-shaped; defining an elongate body section and a generally transverse head, and includes a first side that is intended to contact the bone and a second side opposite the first side. The body section includes a plurality of screw holes for the extension of bone screws therethrough, and, optionally, one or more substantially smaller K-wire alignment holes. The body section may have a gentle S-shape and a proximal twist. Furthermore, the contoured plate provides a stable shape that prevents rocking of the plate on the bone and maintains anatomical alignment between the fracture fragments. The body section of the plate may also be thinner proximally to allow room to avoid the bicep tendon. An advantage of the elongate body is to enable fixation of complex fractures of the proximal radius by providing sufficient purchase of the plate on stable bone structure.

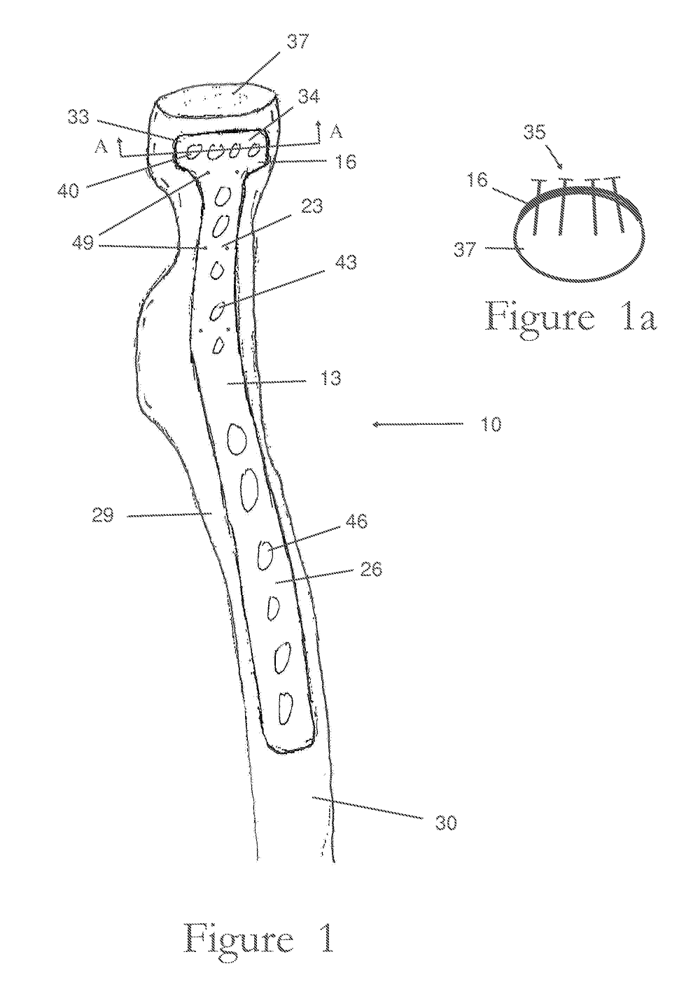

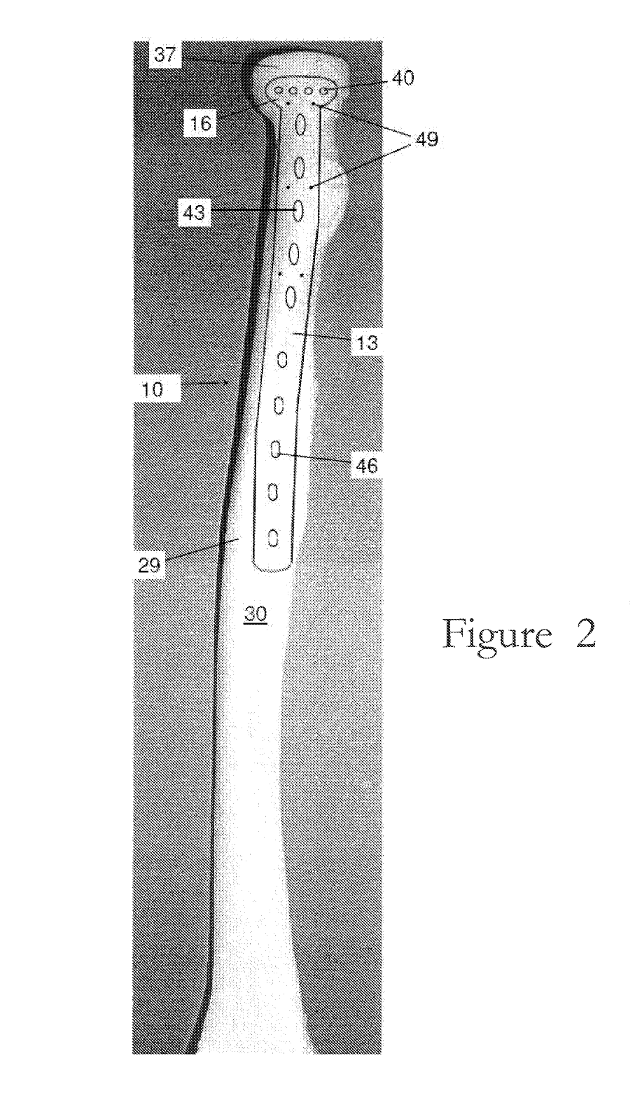

Referring to the drawings, FIGS. 1 and 2 show a proximal radius locking plate, indicated generally as 10, according to the present invention. The plate 10 includes an elongate body 13 and a transverse head 16. The elongate body 13 comprises a first section 23 and a second section 26. The first section 23 is connected on a proximal end to the transverse head 16. In one embodiment, the dimension of the overall length of the elongate body 13 is significantly longer than the dimension of the width of said elongate body 13. Additionally, the elongate body 13 is thinner proximally to allow for the bicep tendon. The elongate body 13 is adapted for receiving bone screws to fix the locking plate 10 to a shaft 29 of the radius 30. The transverse head 16 includes a first head section 33 and a second head section 34. The first and second head sections 33, 34 extend generally perpendicular and laterally away from a longitudinal axis of the first section 23 of the elongate body 13 in opposite directions, overall forming a T-shape. The transverse head 16 is adapted for receiving bone screws 35 configured and arranged to secure a plurality of bone screws 35 divergently implanted in the head 37 of the proximal radius (FIG. 1a).

The locking plate 10 has a plurality of openings. In some embodiments, openings for the extension of bone screws 35 therethrough are provided in at least the transverse head 16 and the two sections 23, 26 of the elongate body 13. A first set of openings 40 is located in the transverse head 16. The openings 40 in the transverse head 16 are adapted for receiving bone screws 35 in the proximal radial head 37. Preferably, though not necessarily, such bones screws 35 are installed in a divergent manner. Such bone screws 35 may comprise 2.4 mm screws. Other sizes may be used. The screws may be locking screws or other appropriate types of screws. Optionally, one or more substantially smaller K-wire alignment holes 49 may also be provided in the transverse head 16 and/or the elongate body 13. A second set of openings 43 is located in the first section 23 of the elongate body 13. The openings 43 in the first section 23 are adapted for receiving bone screws in the bone shaft 29. Such bone screws may comprise 2.7 mm screws. Other sizes may be used. These may be locking screws or other appropriate fasteners. A third set of openings 46 is located in the second section 26 of the elongate body 13. The openings 46 in the second section 26 are adapted for receiving bone screws in the bone shaft 29. Such bone screws may comprise 3.5 mm screws. Other sizes may be used. These screws may be locking screws, combination screws, or other appropriate fasteners. In some embodiments, the openings 43, 46 may comprise slots and are adapted for receiving appropriate fasteners in the radial shaft 29. The openings 40, 43, 46 are sized and shaped for the size and type of the fastener to be used, which selection of size and type is considered within the scope of knowledge of one skilled in the relevant art.

The plate 10 may have a gentle S-shape and a proximal twist to match the contour of the radius. In particular, the plate 10 can be shaped for use in the right arm or the left arm. The plate 10 is pre-shaped to match the contour of the anatomic shape of an unfractured proximal radius 30 and to pass in the region of the radial tuberosity. The elongate body 13 of the locking plate 10 may also be thinner proximally to allow room for the bicep tendon.

In use, the proximal radius locking plate 10 is used for stabilization of fractures of the proximal radius and the radial head. The elongate body 13, with the transverse head 16 attached, is positioned on the radius 30 across the fracture. A drill, or other appropriate device, is used to make holes in the shaft 29 and radial head 37 of the bone 30. Appropriate fixing devices, such as surgical screws, sized and configured for use as described, are inserted transversely of the elongate body 13 and transverse head 16, and pass through at least part of the bone 41 to be treated; the line of insertion of the fixing devices being defined by the openings 40, 43, and 46 in the transverse head 16 and the elongate body 13. Optionally, one or more K-wires may also be used.

### INDUSTRIAL APPLICABILITY

The present invention is applicable to providing a device for stabilizing fractures of the proximal radius. The invention discloses an anatomically shaped bone plate that is pre-shaped to match the contour of the anatomic shape of an unfractured proximal radius in order to assist in reduction of complicated fractures and to avoid bending and cutting of a conventional plate, which can result in a fatigue failure and fracture of the plate.

What is claimed is:

1. A proximal radius locking plate, comprising:
an elongate body having a plurality of openings adapted for receiving bone screws therethrough; and
a transverse head on an end of said elongate body, said transverse head having a plurality of openings adapted for receiving bone screws therethrough;
wherein said plate is sized and configured for securing to a radial head and shaft of a proximal radius.

2. The locking plate according to claim 1, said transverse head comprising:
a first portion extending laterally outward from and generally perpendicular to a longitudinal axis of the elongate body; and
a second portion that extends laterally outward from a longitudinal axis of the elongate body portion in a generally opposite direction than the first portion.

3. The locking plate according to claim 2, wherein said plate is generally T-shaped.

4. The locking plate according to claim 1, wherein said transverse head is configured and arranged to secure a plurality of bone screws implanted in the radial head.

5. The locking plate according to claim 4, wherein said transverse head is configured and arranged to secure said plurality of bone screws divergently in the radial head.

6. The locking plate according to claim 1, said plate being shaped in a gentle S-shape.

7. The locking plate according to claim 1, said plate having a proximal twist.

8. The locking plate according to claim 1, said plate being pre-shaped to match the contour of the anatomic shape of an unfractured proximal radius.

9. The locking plate according to claim 1, said elongate body being thinned proximally to allow for a bicep tendon.

10. The locking plate according to claim 1 wherein the dimension of the overall length of the elongate body is significantly longer than the dimension of the width of said elongate body.

11. The locking plate according to claim 1, said transverse head further comprising a plurality of openings adapted for receiving K-wire therethrough.

12. The locking plate according to claim 1, said elongate body further comprising a plurality of openings adapted for receiving K-wire therethrough.

13. A method of stabilizing a proximal radial fracture, comprising:
providing a locking plate comprising:
an elongate body having a plurality of openings adapted for receiving bone screws therethrough; and
a transverse head on an end of said elongate body, said transverse head having a plurality of openings adapted for receiving bone screws therethrough;
wherein said plate is sized and configured for securing to a radial head and shaft of a proximal radius;
aligning said plate across the proximal radial fracture;
forming a plurality of holes in the radius; and
anchoring the locking plate to the radius using a plurality of bone screws.

14. The method according to claim 13, said locking plate being pre-shaped to match the contour of the anatomic shape of an unfractured proximal radius.

15. The method according to claim 13, said locking plate being shaped in a gentle S-shape.

16. The method according to claim 13, said locking plate having a proximal twist.

17. The method according to claim 13, said elongate body being thinned proximally to allow for a bicep tendon.

18. The method according to claim 13, wherein said transverse head is configured and arranged to secure a plurality of bone screws implanted in the radial head.

19. The method according to claim 13, wherein the step of anchoring the locking plate to the radius using a plurality of bone screws further comprises the step of implanting a plurality of bone screws divergently in the radial head.

20. The method according to claim 13, wherein said transverse head and/or the elongate body further comprise a plurality of openings adapted for receiving K-wire therethrough, said method further comprising the steps of:
reducing said fracture; and
holding the locking plate in position using K-wire.
